(19) United States
(12) Patent Application Publication
(10) Pub. No.: US 2023/0077450 A1
(43) Pub. Date: Mar. 16, 2023

(54) DENTAL SCALER

(71) Applicant: Larissa Ramos, Bainbridge Island, WA (US)

(72) Inventor: Larissa Ramos, Bainbridge Island, WA (US)

(21) Appl. No.: 17/942,890

(22) Filed: Sep. 12, 2022

Related U.S. Application Data
(60) Provisional application No. 63/242,656, filed on Sep. 10, 2021.

(51) Int. Cl. A61C 17/20; A61C 1/00

(52) U.S. Cl. CPC A61C 17/20; A61C 1/0046; A61B 2217/007

(57) ABSTRACT

Described herein are an ultrasonic dental scaler and laser systems. The dental scaler system includes a handpiece and a control unit. The handpiece has a tip, and a water irrigator for providing water to the tip, the handpiece being constructed and arranged to vibrate the tip. In one embodiment the base unit has a dial to control the water flow from the water irrigator to the tip. Also disclosed is a dental laser system which can operate both in a wired and wireless configuration and a root beer concentrate infused dental floss.

### PRIORITY

This patent application claims priority under 35 US § 119(e) to U.S. provisional patent application Ser. No. 63/242,656, filed Sep. 10, 2021, and entitled “Dental Scaler”, the contents of which are incorporated herein fully by reference,

### BACKGROUND

Dental scalers are used to remove plaque buildup and calculus from teeth. Traditionally, dentists have used simple scaler instruments to manually remove plaque and calculus from a patient's teeth. More recently, ultrasonic dental scalers, which utilize a tip that vibrates or oscillates at high speeds, typically ultrasonic speeds, have been developed. The ultrasonic dental scaler tip is applied to a tooth and the tip's oscillations remove the plaque and calculus buildup from the tooth. With existing dental scalers, a base unit is used to control both the intensity of the tip's oscillations and the water flow, for cooling purposes, from a nozzle in a tip portion of the handpiece that includes the tip. However, this arrangement requires the dental scaler operator to turn away from a patient to adjust the volume of water flow or power to the scaler, resulting in taking the focus away from the job at hand and leading to imprecise adjustments of the controls, and poor experience for both the operator and the patient. Foot operated pedals and similar controls have also been considered. However, cabled versions of these pedals are difficult to reposition during therapy if, for example, the operator needs to change positions relative to the patient and can present an entanglement and trip hazard even where repositioning is not required. Further, wireless pedal controls must be frequently charged and synchronized with a scaler base unit transceiver, which can also complicate set-up and operation.

### SUMMARY

According to one aspect of the disclosed subject matter, an ultrasonic dental scaler comprises a handpiece and a base unit. The handpiece includes a tip portion that includes a tip and a nozzle for providing or spraying water to the tip for cooling purposes. The handpiece is coupled to the base unit and configured to provide power to the tip portion which, in turn, causes the tip to oscillate. Additionally, the handpiece includes a user-manipulable control that controls an amount of water provided to the tip portion for cooling. Placing a user-manipulable control on the handpiece to control the amount or volume of water used to cool provides a better experience for the operator and the patient.

Alternatively, or in addition thereto, the handpiece itself may include a user-manipulable control that controls the intensity of the tip oscillation, thereby affording convenient operation of the dental scaler from the handpiece. Also, the handpiece may optionally include a power switch to turn the dental scaler system off or on.

The disclosed subject matter also includes a treatment method which utilizes a dental scaler handpiece that includes both an oscillation intensity or power level control unit and a water flow rate control unit on or as part of the handpiece.

Another aspect of the disclosed subject matter is directed to a root beer infused dental floss created by placing dental floss in a substantially liquid root beer concentrate for a period of time based on the absorbency of the dental floss material and desired degree of flavor. The root beer concentrate may be formed by combining substantially equivalent portions of sarsaparilla, sassafras, licorice root and burdock root.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features. In some instances, well-known methods, procedures, and components have not been described in detail to avoid obscuring the embodiments described.

FIG. 1 illustrates a dental scaler handpiece comprising, at least, a water flow control located on the handpiece, with the handpiece connected to a base unit, in accordance with some embodiments of the disclosed subject matter.

FIG. 2 illustrates a dental scaler handpiece comprising, at least, a water flow control and an on/off control located on the handpiece, with the handpiece connected to a base unit, in accordance with some embodiments of the disclosed subject matter.

FIG. 3 illustrates a dental scaler handpiece comprising, at least, a water flow control, an on/off control, and a power level control for the scaler tip, with the handpiece connected to a base unit (no control on the base unit except another power switch), in accordance with some embodiments of the disclosed subject matter.

FIG. 4 illustrates a dental laser tool comprising, at least, a laser emitting tip and an on/off control, with the handpiece connected to a base unit, in accordance with some embodiments of the disclosed subject matter.

FIG. 5 illustrates a dental laser tool connected to a base unit, the laser tool includes an on/off control and a control to vary the power delivered to the tool (no control on the base unit except another power switch), in accordance with some embodiments of the disclosed subject matter

FIG. 6 illustrates a combined laser/scaler base unit with the connected laser and scaler tools, each tool with an on/off switch and the scaler tool with a water flow control, and a power level control, in accordance with some embodiments of the disclosed subject matter.

FIGS. 7A-7C show a dental scaler handpiece according to further embodiments of the disclosure that includes in-line twistable barrel selectors for oscillation intensity and water flow.

FIG. 8 is a flowchart describing the treatment method using the dental scaler system shown in FIG. 3.

### DETAILED DESCRIPTION

The following describes an ultrasonic dental scaler that includes a base unit and a handpiece, formed in accordance with aspects of the disclosed subject matter. The handpiece comprises a barrel portion and a tip portion, where the tip portion includes a tip that, when powered, oscillates. The tip portion further comprises a nozzle for spraying water on the tip, for cooling purposes. The barrel portion of the handpiece is typically, though not exclusively, a slender object. The barrel portion may be, by way of illustration and not limitation, triangular, oval, rectangular, cylindrical, or some other shape that it can be easily held by an operator and used for inserting and manipulating the tip portion in the mouth cavity of a patient for cleaning. The handpiece further comprises a first user-manipulable control, i.e., a water flow control, which controls the volume of water sprayed by the nozzle to cool the tip. This water flow control may, in some embodiments, incorporate a barrel-mounted dial to permit easy adjustment of the water flow rate without the operating oral care provider from having to turn away from the patient. In some embodiments, the handpiece may further comprise a second user-manipulable control that controls the intensity of the oscillations of the tip, as described later in this section.

Traditionally, dentists and other oral healthcare professionals have utilized simple dental scaler instruments to remove plaque and calculus from a patient's teeth. More recently, however, ultrasonic dental scalers have been developed. The ultrasonic dental scaler tip is applied to a tooth and the oscillations of the tip cause the plaque and calculus buildup to be removed from the tooth. In existing solutions, a foot manipulated base unit is used to control both the intensity of the tip's oscillations and the water flow volume from tip. However, manipulating the controls of the base unit requires the dental scaler operator to turn away from the patient to adjust the water flow and/or power to the scaler, resulting in taking the focus away from the job at hand and leading to imprecise adjustments of the controls, and poor experience for both the operator and the patient.

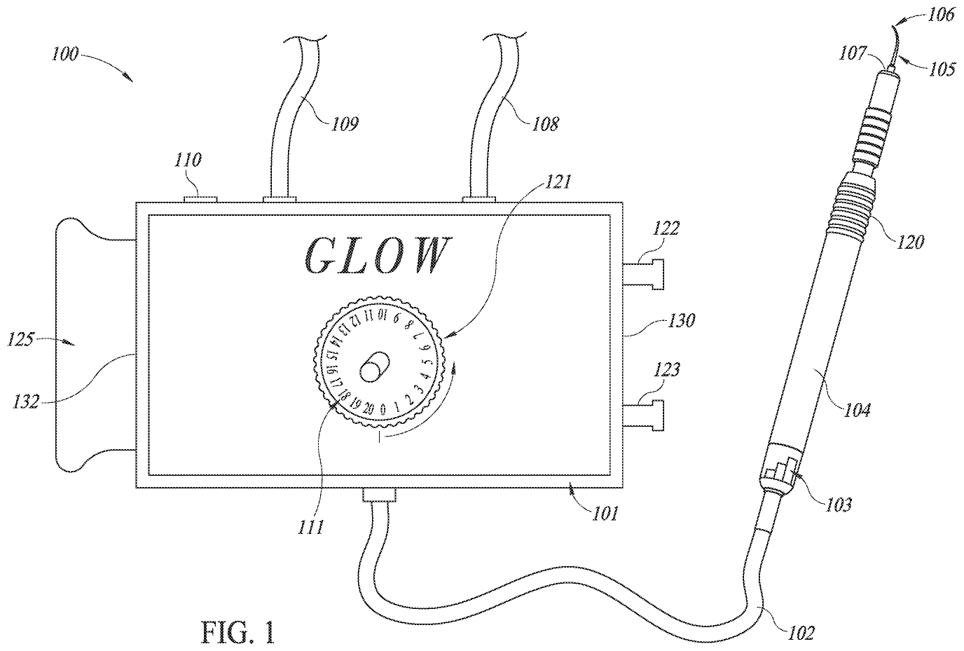

In the illustrated embodiment shown in FIG. 1, the dental scaler system (100) comprises a base unit 101 and a handpiece 104. The handpiece 104 further comprises a tip 105 with a nozzle 106, and a water flow control unit incorporating dial 103. A coupling 102 between the base unit and the ultrasonic dental scaler includes a water hose as well as an insulated power connection that electrically connects a distal end of the handpiece 104 to the base unit 101. Note here that the tip 105 can be illuminated by an LED or LEDs assembly 107 to assist the oral care provider with moving the tip 105 and handpiece 104 to more effectively contact and treat the tooth.

Also shown in on handpiece 104 are plural raised bump grips 120 to allow the oral care provider's hand to firmly grasp and manipulate the handpiece 104.

In the illustrated embodiment, the base unit 101 includes a user-manipulable power level control 111 that, in some non-limiting embodiments, may be a dial. Here, the dial is substantially circumscribed by a ridged edge ring 121 made of a transparent material. This transparent ring 121 can be illuminated by e.g. an LED, LED array or other suitable light pipe guiding light from a light source (not shown) within the base unit 101to selectively highlight the current dial setting. The base unit 101 also includes a connection 108 to a power source such as a power cord and a water supply line 109 from the water mains. The base unit 101 supplies power to electronic components of the dental scaler handpiece 104 which, in turn, delivers power to the tip 105 causing oscillations and enabling water to be sprayed from a nozzle 106 to cool the tip.

Also shown in FIG. 1, the base unit 101 also includes cradle arms 122, 123 extending from at least one side 130 of the base unit to releasably secure and store the scaler handpiece 104 when not in use. FIG. 1 also depicts a charging cradle 125 on the opposing side 132 of the base unit from the base unit side 130 and the cradle arms 122, 123. This charging cradle can be used to store and charge, for example, a cordless laser treatment apparatus (not shown).

With the illustrated embodiment, when operating the dental scaler system 100, an operator removes plaque and calculus from a tooth by touching the tip 105 to the plaque and calculus on the tooth surface.

As those skilled in the art will appreciate, the high frequency oscillations of the tip 105 may cause the tip to heat up. The operator cools the tip by controlling the flow of water provided by the coupling 102 to the nozzle 106 which may be located on or near the tip 105. The volume of water used for cooling the tip 105 may be controlled by a water flow control unit 103 which, advantageously, is also located on the handpiece 104. Though not intended to be limiting the teachings of the disclosed embodiments, this water flow control 103 may include, for example, a handpiece barrel mounted water lavage control assembly provided with the Cavitron Select SPS Ultrasonic Scaler from Dentsply Sirona of Charlotte, N.C. As the heat increases on the ultrasonic dental scaler tip 105, perhaps from extended use, the operator may increase cooling of the tip by increasing the water flow to the tip 105 via manipulation of the water flow control, and as the heat decreases, the operator may decrease the water flow to tip 105 via manipulations of the water flow control. The base unit is shown to include a power switch 110 that serves as an on/off control for the ultrasonic dental scaler system 100.

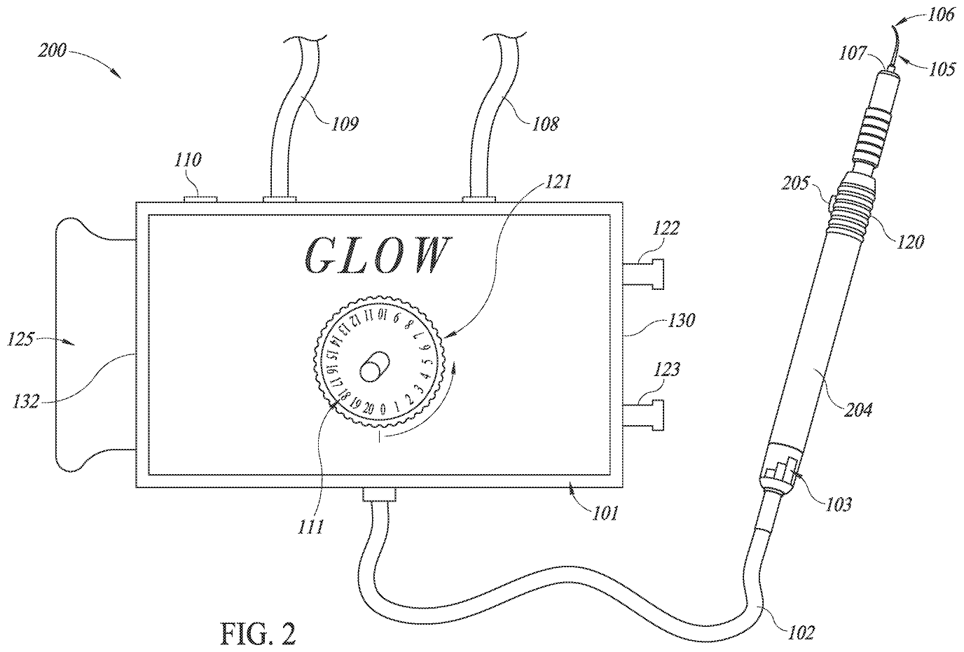

FIG. 2 shows an alternative ultrasonic dental scaler system 200 embodiment where the handpiece 204 of the ultrasonic dental scaler system houses a moveable or depressible (momentary and/or lockable) power switch 205. The handpiece 204 in FIG. 2 differs, at least in part, from the handpiece 104 in FIG. 1 in the inclusion of this power switch 205. The inclusion of the power switch 205 on the handpiece allows the operator a convenient way to turn or power off/on the dental scaler system 200 from the handpiece 204 instead of having to turn attention away from the patient and accessing the same control on the base unit 101 as shown in FIG. 1. The state of this power switch 205 can be communicated or assessed through the electrical connection made between control circuitry in the base unit 201. Alternatively, though not shown in the figures, the state of the power switch 205 may be communicated over a wireless communications link, such as a Bluetooth LE, WiFi or similar link and protocol, as will be understood by those ordinarily skilled in the art. The base unit 201 in FIG. 2 differs, at least in part, from the base unit 101 in FIG. 1 in that it has the ability to determine the state of the power switch 205 and throttle power accordingly to the handpiece 204.

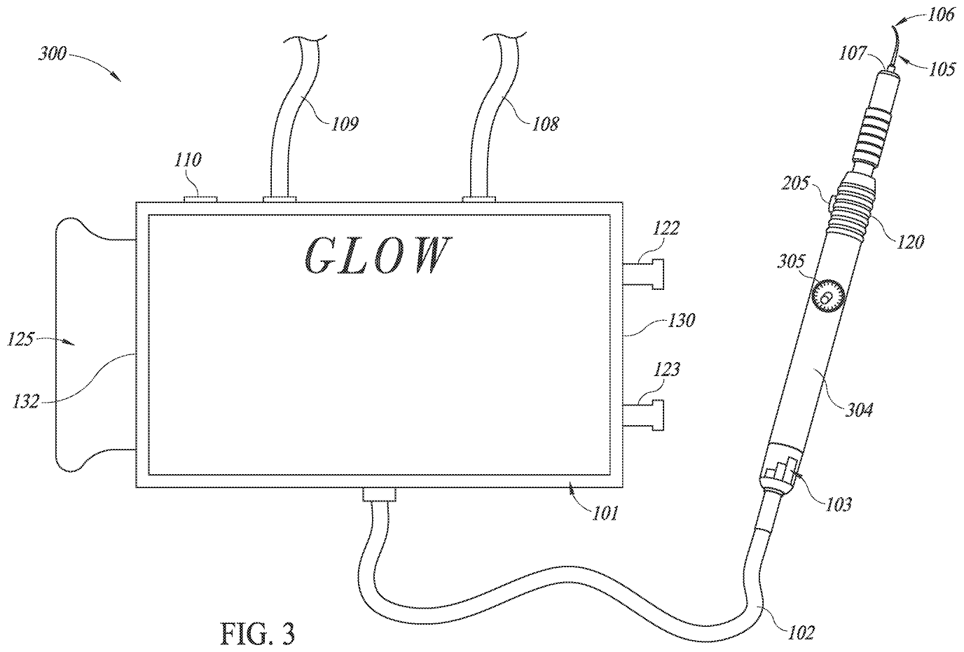

Another problem with existing dental scalers where the power-level is adjusted via a foot control is that if the operator wants to control the amount of power supplied to the tip, i.e., wishes to control the intensity of oscillations by the tip, the operator must stop the treatment, turn away from the patient and adjust an appropriate control, e.g., a dial, on the base unit. This takes the focus away from the patient and may result in imprecise adjustments to the power level. To remedy this problem, we refer to the embodiment shown in FIG. 3. In FIG. 3, the handpiece 304 of the ultrasonic dental scaler system 300, in addition to comprising the components on the handpieces 104 and/or 204 shown in FIGS. 1 and 2 respectively, also has an illuminated power level control 305 for manipulating the intensity of the oscillations of the tip 105 on the handpiece 304. As shown in FIG. 3, this may be implemented in the form of an illuminated dial communicating with, for example, oscillation intensity control circuitry including a variable potentiometer, step motor or similar as will be understood by those ordinarily skilled in the art to vary and control the intensity and/or frequency of the ultrasonic oscillations of the tip 105. This affords the operator of the dental scaler a convenient method to review status of and/or manipulate all the three controls on the handpiece 304—the water flow level control 103 to adjust the volume of water delivered to the tip, the power level control 305 to adjust the intensity of the oscillations on the tip 105, and the power switch 205 to turn the dental scaler system 300 off or on. The base unit 301 in FIG. 3 differs, at least in part, from the base unit 201 in FIG. 2 in that the base unit 301 no longer includes a power level control.

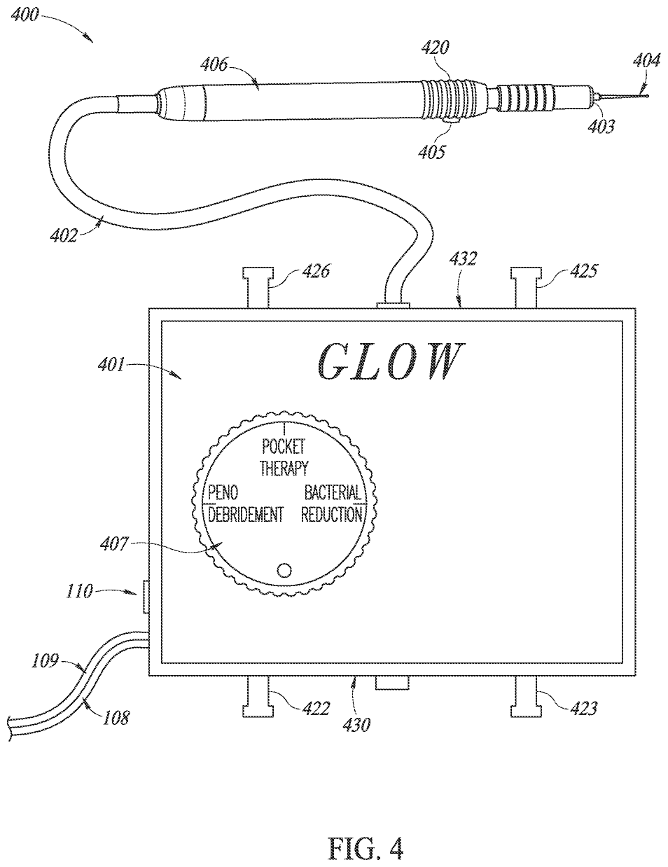

According to additional aspects of the disclosed subject matter a dental laser system 400 (FIG. 4) is presented. The dental laser system 400 is used in performing dental procedures involving the soft tissues, teeth, and bone in the oral cavity. FIG. 4 shows a dental laser system 400 having a handpiece 406 connected to a base unit 401. The handpiece 406 comprises, at least, a laser emitting diode 404 at a removable tip of the handpiece. There is also a tip illumination LED 403 at the base of the tip. The handpiece 406 is coupled to a base unit 401 via a coupling 402. The base unit 401 among other things, provides power to the laser via the coupling 402. The handpiece 406 also has a power switch 405 on its body to allow the operator to turn the dental laser system 400 on or off from the handpiece. In some alternative embodiments, the handpiece 406 may include a battery to provide power to the laser, independent of power from an outside source or from the base unit 401. The base unit 401 has a power level control 407 to adjust the intensity of the power provided to the laser emitting diode 404 at the tip of the handpiece. In the non-limiting illustrated embodiment shown in FIG. 4, the power level control 407 provides three preset levels of intensity selection—a bacterial reduction level, a pocket therapy level, and a perio debridement level.

As with the dental scaler handpieces 104, 204 and 304 described above, the periodontal laser handpiece 406 includes bump grips 420 to help the oral care provider grasp and manipulate the handpiece 406 during treatment.

FIG. 4 also depicts the dental scaler handpiece rests 422, 423 on one side 430 of the base unit 401. On the opposing side 432 of the base unit 401, rests 425, 426 extend outwardly therefrom to releasably support and secure the periodontal laser handpiece 406 when not in use. The base unit also includes a unit on/off power switch 110, as well as water supply line 109 and power cord 108 ports.

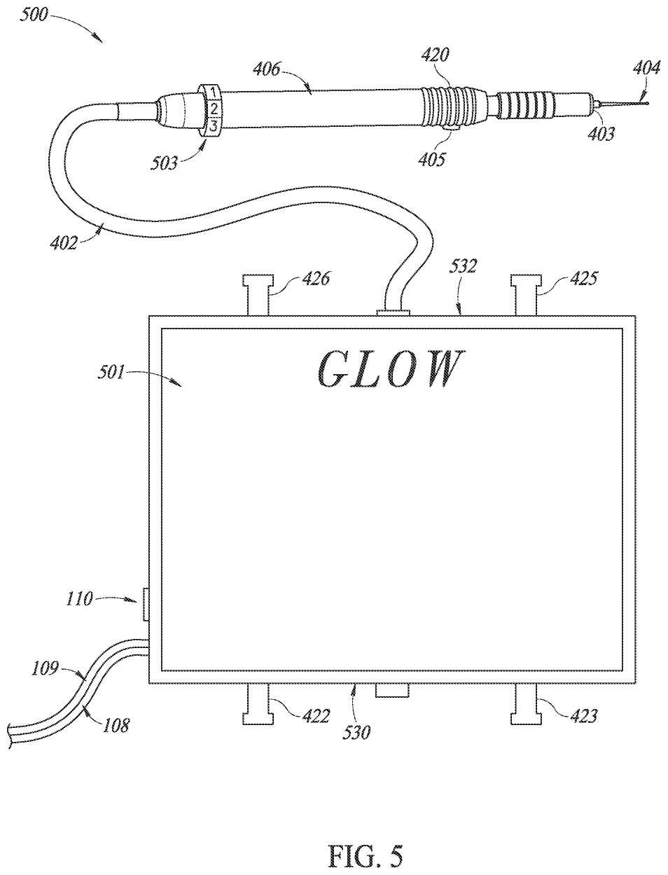

FIG. 5 shows an alternative embodiment, a dental laser system 500, where the dental laser's handpiece 506, in addition to housing the power switch control 405, also houses a twistable, barrel-mounted power level control 503 to help the operator adjust the intensity of the laser emitting diode 404. In some embodiments, the power level control 503 provides the user with the same three levels of intensity selection as power level control on the base unit 401 shown in the embodiment in FIG. 4—a bacterial reduction level, a pocket therapy level, and a perio debridement level. The base unit 501 in FIG. 5 differs from the base unit 401 in FIG. 4 in that it no longer includes the laser power level control.

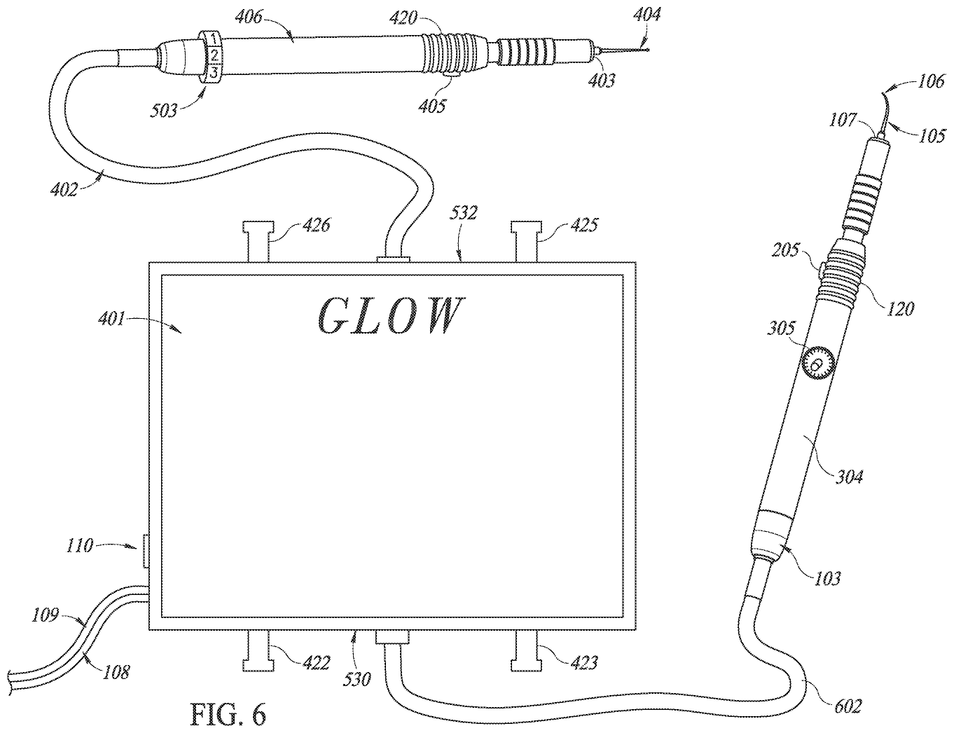

FIG. 6 shows another alternative embodiment of a dental scaler and laser system 600 including both, a dental scaler handpiece 304 connected to a base unit 601 via a first coupling 602, and a dental laser handpiece 506 connected to the base unit 601 via a second coupling 402.

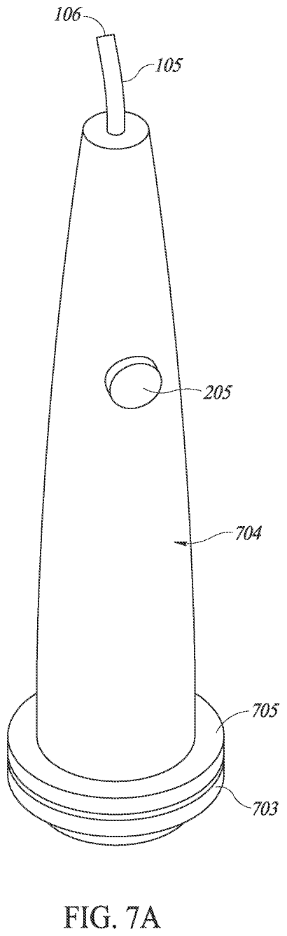

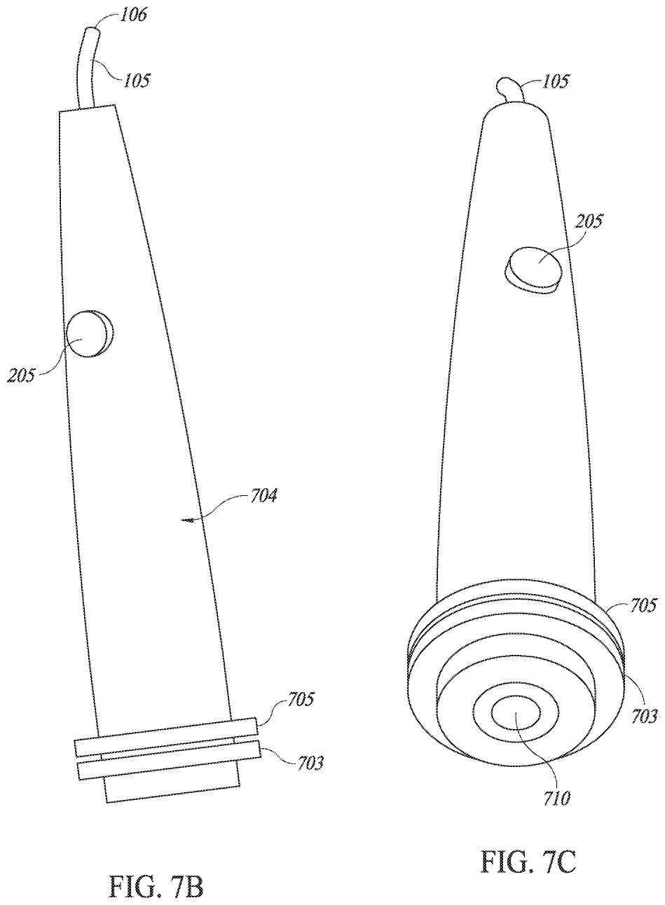

FIGS. 7A-C depict an alternative embodiment of a dental scaler handpiece portion 704 according to further embodiments of the disclosure. In this embodiment, the water/electrical coupling 102 connecting the handpiece to the base is not shown save for a coupling interface 710 (FIG. 7C) terminating at the handpiece bottom surface. The handpiece 704 includes two hand twistable barrel-style selector control units with dials 703, 705 that circumscribe the handpiece 704 body. Of the two selector control unit dials, dial 703 located nearest the distal end of the handpiece 704 is configured to be used by the oral care provider to select the water flow rate similarly to the water flow control unit 103 described previously. Barrel dial 705 interposing the water flow control unit dial 703 and the tip is used to adjust the oscillation intensity of the scaler tip 105 through a range of power levels.

In FIGS. 7A-7C, power level dial 705 is positioned between the tip and the water flow dial 703 and relatively proximate to the water flow dial 703. This configuration allows one-handed adjustment of either the oscillation power level/intensity or the water flow rate, or both as needed using an efficient one-handed movement by the oral care provider while the provider's other hand continues to grasp the handpiece 704 and can apply the tip 105 to the patient.

Although the previous embodiments describe the water flow rate and power level/intensity controls in terms of adjustable or twistable dials, one or more other types of selector mechanisms can be interchangeably provided, including one or more multi-position or overloaded buttons, sliders and/or the like on the dental scaler or laser handpiece so long as they can provide settings feedback to the operator and can be manipulated without having to turn away from the patient.

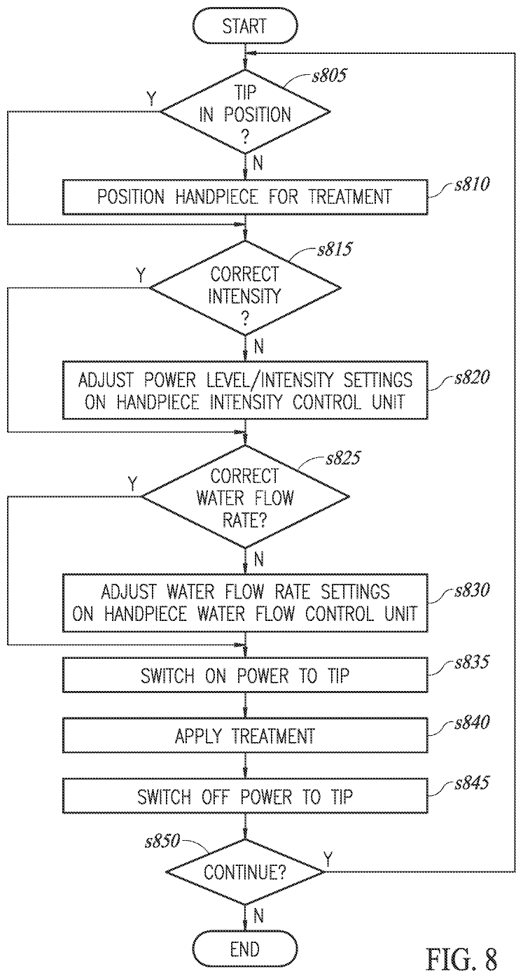

FIG. 8 illustrates an embodiment of a treatment method which utilizes a dental scaler such as dental scaler 300 shown in FIG. 3. This method begins at step s805, in which a determination is made whether the scaler tip of the tip portion 105 is positioned correctly for treatment. This is typically done by observation and/or feel based on the experience of the oral care treatment provider. If, in step s805 it is determined that the tip is not in the correct position, the method progresses to step s810 where the treatment provider moves the handpiece 304 to correct the tip position for treatment. Once in the correct position, the method progresses to step s815 If, however, in step s805 it is determined that the tip is in the correct position, the method instead jumps to step s815.

At step s815, a determination is made by e.g. a treatment provider whether the dental scaler 300 is set at the correct oscillation intensity or power level for the scaler tip. In this embodiment, the treatment provider can look at the current setting on control unit 305 dial on the handpiece 304. If the setting is incorrect, the method progresses to step s820, where the treatment provider adjusts the power level/intensity setting on the scaler handpiece 304 using the control unit 305 dial. Once adjusted to the correct setting, the method progresses to step s825. If, however, in step s815, it is determined that the correct power level/intensity is already set, the method instead jumps to step s825.

At step s825, a determination is made e.g. by the treatment provider whether the dental scaler system 300 is set at the correct water flow rate. In this embodiment, the treatment provider can assess the current water flow rate from viewing the current position or state of the water flow rate control unit 103 (dial) on the handpiece 304. If the water flow rate control unit is not correctly set, the method progresses to step s830. At step s830, the treatment provider adjusts the water flow rate control unit 103 settings on the handpiece 304. Once the correct water flow rate is set, the method then progresses to step s835. If, however, in step s825, it is determined that the water flow rate is correct, the method instead jumps to step s835.

At step s835, the treatment provider switches on the power to the tip using, in this embodiment, switch 205 shown in FIG. 3. Then, at step s840, the provider uses the handpiece to apply treatment to the patient's teeth. After completion of treatment (or at e.g. a suitable stopping point), the treatment provider then switches off power to the scaler tip in step s845. The method then progresses to step s850.

At step s850, a determination is made whether treatment should continue (such as treating another tooth). If so, the method jumps back iteratively to step s805. If, however, treatment is completed or otherwise stopped, this treatment method ends.

Note that although it is contemplated that in this embodiment, for example determining steps s805, s815, s825 and s850 shown in FIG. 8 are carried out by an oral care treatment provider such as a dentist or dental hygienist, these steps could be undertaken in different ways including, without limitation, through the use or assistance of a special purpose or general purpose computer or information processing system programmed in accordance with one or more steps of this method. Moreover, the specific order of the steps of this method need not be limited to the embodiment of FIGS. 8 and 3, so long as the water flow rate and the scaler tip power level/intensity can be controlled from the dental scaler handpiece.

A further embodiment of the disclosed subject matter is directed to natural root beer infused dental floss. This floss involves dental floss (e.g. polyester and/or cotton filaments) soaked in a unique substantially liquidous root beer concentration as described below for approximately one (1) hour. In other embodiments, this soaking time may be longer or shorter based on e.g., the desired infusion of the root beer concentrate, the absorbency of the dental floss material. Once soaking is complete, the root beer “flavor” of the dental floss can be maintained by coating the treated dental floss with coconut oil.

The root beer concentration used here involves the following:

Ingredients: 4½ cups water, 1 large thumb of thinly sliced ginger root, 2½ tbsp of sarsaparilla, 2½ tbsp of sassafras, 2½ tbsp of licorice root, 2½ tbsp of burdock root, 1 tbsp of dried mint leaves, 1 star anise pod, 1 vanilla bean, and ½ cup of xylitol.

Instructions for making the root beer concentration: combine all ingredients except xylitol in a saucepan and bring to a simmer for 30 minutes. Thereafter, stir in xylitol until dissolved. Simmer this mixture on very low heat for an additional 30 minutes. Strain out the bits from the resulting mixture and retain the liquid. Then allow the liquid to cool to room temperature and refrigerate before use.

### CONCLUSION

Although the subject matter has been described in language specific to features and methodological acts, it is to be understood that the subject matter defined in the appended claims is not necessarily limited to the specific features or acts described herein. Rather, the specific features and acts are disclosed as exemplary forms of implementing the claims.

What is claimed is:

1. An ultrasonic dental scaler comprising a handpiece, coupled to a base unit, and wherein the handpiece comprises:
a barrel portion configured to be grasped in a course of using the dental scaler;
a tip portion connected to an end of the barrel portion and configured to cause a scaler tip extending from the tip portion to oscillate ultrasonically when the tip portion is provided with power;
wherein a coupling between the handpiece and the base unit includes a channel to provide water from the base unit to the handpiece;
a first user manipulable control on the barrel portion of the handpiece that controls an amount of water provided to the handpiece for cooling; and
a second user manipulable control on the barrel portion of the handpiece that controls an intensity of the oscillations of the scaler tip.

2. The ultrasonic dental scaler of claim 1, wherein the first user manipulable control comprises a first twistable barrel-mounted dial.

3. The ultrasonic dental scaler of claim 2, wherein the second user manipulable control comprises a second twistable barrel-mounted dial.

4. The ultrasonic dental scaler of claim 3, wherein the first and second barrel-mounted dials are positioned distally from the tip portion of the handpiece.

5. The ultrasonic dental scaler of claim 1, wherein the second user manipulable control comprises a second twistable barrel-mounted dial.

6. The ultrasonic dental scaler of claim 1, wherein the handpiece further comprises a power switch disposed on a surface of the handpiece configured to selectably control power delivery to at least the scaler tip of the handpiece.

7. The ultrasonic dental scaler of claim 1, wherein the tip portion further comprises at least a first nozzle for spraying the water during treatment.

8. The ultrasonic dental scaler of claim 1, further comprising a dental laser tool coupled to the base unit.

9. An ultrasonic dental scaler handpiece, comprising:
a barrel portion configured to be grasped during treatment;
a tip portion connected to a first end of the barrel portion and configured to cause a scaler tip extending from the tip portion to oscillate ultrasonically when the tip portion is provided with power;
a coupling interface disposed on an external surface of the handpiece substantially opposing the tip portion to accept water to the barrel and tip portions;
a first user manipulable control on the barrel portion that controls an amount of the water provided to at least the tip portion for cooling; and
a second user manipulable control on the barrel portion that controls an intensity of the oscillations of the scaler tip.

10. The ultrasonic dental scaler handpiece of claim 9, wherein the first user manipulable control comprises a twistable barrel-mounted dial.

11. The ultrasonic dental scaler handpiece of claim 10, wherein the second user manipulable control comprises a twistable barrel-mounted dial.

12. The ultrasonic dental scaler handpiece of claim 11, wherein the first and second barrel-mounted dials are positioned distally from the tip portion.

13. The ultrasonic dental scaler handpiece of claim 9, wherein the second user manipulable control comprises a second twistable barrel-mounted dial.

14. The ultrasonic dental scaler handpiece of claim 9, further comprising a third user manipulable control disposed on the barrel portion configured to selectably control power to at least the scaler tip.

15. The ultrasonic dental scaler handpiece of claim 9, wherein the tip portion further comprises at least a first nozzle for spraying the water during treatment.

16. The ultrasonic dental scaler handpiece of claim 9, wherein the first user manipulable control comprises means for controlling the amount of the water provided to at least the tip portion.

17. The ultrasonic dental scaler handpiece of claim 16, wherein the second user manipulable control comprises means for controlling the intensity of the oscillations of the scaler tip.

18. A method, comprising:
positioning a dental scaler handpiece for treatment, the dental scaler handpiece defining a tip portion having an ultrasonic scaler tip;
selectably adjusting an intensity of oscillations at the ultrasonic scaler tip using a first user manipulable control positioned on the dental scaler handpiece; and
selectably adjusting a water flow rate for water provided to the tip portion using a second manipulable control positioned on the dental scaler handpiece.

19. The method of claim 18, further comprising selectably applying power to the scaler tip by a third user manipulatable control position on the dental scaler handpiece.

20. The method of claim 19, wherein the first and second manipulable controls comprise first and second twistable barrel dials circumscribing the dental scaler handpiece.
